(19) United States
(12) Patent Application Publication
(10) Pub. No.: US 2026/0366046 A1
(43) Pub. Date: Nov. 19, 2026

(54) AN AQUACULTURE SYSTEM AND METHODS FOR REMOVING GLYCEROL FROM THE AQUACULTURE SYSTEM

(71) Applicant: Neste Oyj, Espoo (FI)

(72) Inventors: Jeffrey KANEL, Porvoo (FI); Cecil CHURN III, Porvoo (FI); Aino-Maija LAKANIEMI, Porvoo (FI); Perttu KOSKINEN, Porvoo (FI)

(21) Appl. No.: 19/144,096

(22) PCT Filed: Dec. 22, 2023
(86) PCT No.: PCT/FI2023/050729

§ 371 Date: Jun. 27, 2025

Related U.S. Application Data
(60) Provisional application No. 63/477,887, filed on Dec. 30, 2022.

(30) Foreign Application Priority Data
Mar. 21, 2023 (FI) 20235331

(51) Int. Cl. C12P 7/20; C12M 1/00; C12M 1/26; C12N 1/12

(52) U.S. Cl. CPC C12P 7/20; C12M 23/18; C12M 23/38; C12M 33/00; C12M 47/10; C12N 1/12

(57) ABSTRACT

The present disclosure relates to methods for removing glycerol from an aquaculture system. These methods can include: culturing a first population of microorganisms that produce glycerol in a first reaction zone, thereby providing an aqueous stream containing a microbial biomass material that includes glycerol; inputting the aqueous stream containing the microbial biomass material that includes glycerol to a harvesting zone; separating the microbial biomass from the glycerol in the harvesting zone, thereby providing an aqueous glycerol stream; supplying the aqueous glycerol stream to a second reaction zone containing a second population of microorganisms that metabolize glycerol; and removing glycerol from the aqueous glycerol stream by cultivating the second population of microorganisms in the second reaction zone with the aqueous glycerol stream, thereby providing an enriched aqueous stream containing glycerol metabolites. Systems for performing the above methods are also provided.

### FIELD

The present disclosure relates to an aquaculture system and methods for removing glycerol from the aquaculture system. In exemplary embodiments, the aquaculture system contains algae, bacteria, archaea, or any combination thereof that produce valuable biological products.

### BACKGROUND INFORMATION

There is an increasing interest in using algal biomass as a key intermediate for a plethora of sustainable products, such as a source of renewable energy, as a mode to safely and efficiently capture carbon dioxide from the atmosphere for carbon sequestration, as a source of natural pigments, such as carotenoids, and as a renewable source of chemical intermediates. The production of an algal biomass can be achieved through a variety of methods, some of which involve using an aquaculture system of algae, bacteria, archaea or combinations thereof. In these systems, the algal biomass produced from the aquaculture of algae, bacteria, archaea or combinations thereof can be transported in an aqueous stream to downstream purification units that aid in the removal of the useful biological products from the algal biomass material.

Not only can these aquaculture systems produce an aqueous stream containing the algal biomass material, they also have been shown to produce high levels of glycerol as well. Indeed, for example, aqueous streams originating from aquacultures of various algae of the Dunaliella species have been found to contain glycerol. These types of algae may accumulate, under extreme conditions, up to about 85 percent by weight of glycerol.

These high levels of glycerol can be carried through with the algal biomass material in the aqueous stream to downstream processing units. Once the biomass material has been separated and recovered from the aqueous stream, the glycerol remaining in the aqueous stream may be recovered. This is due to the fact that glycerol can be used as a reactive intermediate or component in the synthesis of other useful biological materials, such as CO₂, lipids, proteins and other macromolecules.

Traditionally, distillation is used to separate glycerol from an aqueous stream but that requires evaporating all of the water which carries the glycerol. The removal of water by this distillation process tends to be expensive, especially when dilute glycerol amounts are present in the aqueous stream. In some cases, salt is also present in the aqueous stream containing the glycerol. In these instances, the salt tends to remain with the glycerol in the reboiler of the distillation unit, thereby making it more difficult to isolate glycerol with a high purity.

Therefore, simple methods that can effectively recover a biomass material and remove glycerol from an aqueous stream without the need to expend large amounts of energy would be a favorable contribution to the art.

To address the foregoing issues, the inventors of the present disclosure have developed a more economically feasible and efficient process for removing glycerol from aqueous streams originating from aquacultures of algae, bacteria, archaea or combinations thereof.

### SUMMARY

A method is disclosed for removing glycerol from an aquaculture system, the method includes at least one or more of the following: (i) culturing a first population of microorganisms that produce glycerol in a first reaction zone, thereby providing an aqueous stream containing a microbial biomass material that includes glycerol; (ii) inputting the aqueous stream containing the microbial biomass material that includes glycerol to a harvesting zone; (iii) separating the microbial biomass from the glycerol in the harvesting zone, thereby providing an aqueous glycerol stream; (iv) supplying the aqueous glycerol stream to a second reaction zone containing a second population of microorganisms that metabolize glycerol; and (v) removing glycerol from the aqueous glycerol stream by cultivating the second population of microorganisms in the second reaction zone with the aqueous glycerol stream, thereby providing an enriched aqueous stream containing glycerol metabolites.

A method for removing glycerol from an aquaculture system, the method comprising: (i) culturing a first population of microorganisms that produce glycerol in a first reaction zone, thereby providing an aqueous stream containing a microbial biomass material that includes glycerol; (ii) inputting the aqueous stream containing the microbial biomass material that includes glycerol to a harvesting zone; (iii) separating the microbial biomass from the glycerol in the harvesting zone, thereby providing an aqueous glycerol stream; (iv) supplying the aqueous glycerol stream to a second reaction zone containing a second population of microorganisms that metabolize glycerol; and (v) removing glycerol from the aqueous glycerol stream by cultivating the second population of microorganisms in the second reaction zone with the aqueous glycerol stream, thereby providing an enriched aqueous stream containing glycerol metabolites; wherein a ratio of eukaryotic algae cells/ml to prokaryote cells/ml is higher than in the first population of microorganisms than in the second population of microorganisms is also disclosed.

An aquaculture system is also disclosed, wherein glycerol is removed from the system, the system including at least one or more of the following: (i) a first reaction zone including a first population of microorganisms that produce glycerol; (ii) a harvesting zone in communication with the first reaction zone, the harvesting zone including a harvester; and (iii) a second reaction zone in communication with the harvesting zone, the second reaction zone including a second population of microorganisms that metabolize glycerol.

An aquaculture system, wherein glycerol is removed from the system, the system comprising: (i) a first reaction zone including a first population of microorganisms that produce glycerol, (ii) a harvesting zone in communication with the first reaction zone, the harvesting zone including a harvester and (iii) a second reaction zone in communication with the harvesting zone, the second reaction zone including a second population of microorganisms that metabolize glycerol, is also disclosed.

A composition is also disclosed which can be produced from any of the methods and/or systems disclosed herein.

Also disclosed is an aqueous glycerol stream, the aqueous glycerol stream being obtained from a method that includes at least one or more of the following: culturing a first population of microorganisms that produce glycerol in a first reaction zone, thereby providing an aqueous stream containing a microbial biomass material that includes glycerol; inputting the aqueous stream containing the microbial biomass that includes glycerol to a harvesting zone; and separating the microbial biomass from the glycerol in the harvesting zone, wherein the aqueous glycerol stream has a dissolved glycerol concentration from about 0.1 to about 20000 ppm.

Also disclosed is use of the aquaculture system of the present invention for removing glycerol from an aqueous glycerol stream.

### BRIEF DESCRIPTION OF THE DRAWINGS

Other features and advantages of the present invention will be apparent to those skilled in the art reading the following detailed description in conjugation with the exemplary embodiments illustrated in the drawings, wherein:

FIG. 1 shows a block flow diagram of an exemplary embodiment wherein the glycerol removal method occurs in an open system.

FIG. 2 shows a block flow diagram of an exemplary embodiment wherein the glycerol removal method occurs in an open system including a hydraulic pump to help transport an aqueous stream containing a microbial biomass material that includes glycerol to a harvester from an aquaculture system of one or more ponds.

### DETAILED DESCRIPTION

Disclosed herein are aquaculture systems configured for performing methods of glycerol removal.

The methods for removing glycerol from the aquaculture systems, can include at least one or more of the following: (i) culturing a first population of microorganisms that produce glycerol in a first reaction zone, thereby providing an aqueous stream containing a microbial biomass material that includes glycerol; (ii) inputting the aqueous stream containing the microbial biomass material that includes glycerol to a harvesting zone; (iii) separating the microbial biomass from the glycerol in the harvesting zone, thereby providing an aqueous glycerol stream; (iv) supplying the aqueous glycerol stream to a second reaction zone containing a second population of microorganisms that metabolize glycerol; and (v) removing glycerol from the aqueous glycerol stream by cultivating the second population of microorganisms in the second reaction zone with the aqueous glycerol stream, thereby providing an enriched aqueous stream containing glycerol metabolites.

In exemplary embodiments, the culturing step involves enhancing growth conditions in the first reaction zone for growth of the first population of microorganisms.

Enhancing growth conditions can include, but is not limited to, increasing/decreasing the amount of carbon dioxide transferred into the first reaction zone, increasing/decreasing the types and amount of nutrients and/or trace elements fed to the first reaction zone, increasing/decreasing the amount of light that the first population of microorganisms receives in the first reaction zone, increasing/decreasing the turbidity of the first reaction zone, increasing/decreasing the salt concentration of the first reaction zone, increasing/decreasing the temperature of the first reaction zone, increasing/decreasing the amount of glycerol produced by the first population of microorganisms, increasing/decreasing total organic carbon, lowering the concentration of dissolved glycerol in the first reaction zone and/or any combination thereof.

In exemplary embodiments, the cultivating step involves enhancing growth conditions in the second reaction zone for growth of the second population of microorganisms.

Enhancing cultivation conditions can include increasing/decreasing the amount of oxygen or air transferred into the second reaction zone, increasing/decreasing the types and amount of organic compounds (e.g., acetate, alcohols and/or sugars) fed to the second reaction zone to boost the second population of microorganisms to degrade or metabolize glycerol, increasing/decreasing the types and amount of nutrients and/or trace elements fed to the second reaction zone, increasing/decreasing the amount of light that the second population of microorganisms receives in the second reaction zone, increasing/decreasing the turbidity of the second reaction zone, increasing/decreasing the salt concentration of the second reaction zone, increasing/decreasing the temperature of the second reaction zone, and/or any combination thereof.

In exemplary embodiments, the method for removing glycerol from an aquaculture system includes adding an aqueous solution from a water source to the first reaction zone.

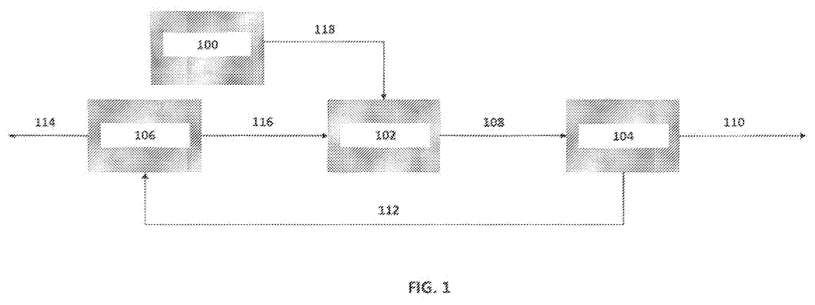

An exemplary embodiment of a method for removing glycerol from an aquaculture system is depicted in FIG. 1. This method involves an aquaculture system containing a water source (100), a first reaction zone (102) containing of one or more ponds with an aqueous environment containing a first population of microorganisms capable of producing glycerol, a harvesting zone (104) and a second reaction zone (106). In this embodiment, an aqueous solution is added into the first reaction zone (102) from the water source (100).

The water source can include, but is not limited to, tubular reactors, photobioreactors, enclosed raceways, covered ponds, open raceways, open ponds, earthen ponds, ponds in greenhouses, clear plastic bags hung either indoors or outdoors, fermenters, naturally occurring bodies of water, solar salt ponds, and combinations thereof. Any design of a fermenter, pond, or bioreactor known in the art that can provide an aqueous solution capable of growing microorganisms that produce glycerol is an acceptable water source. In some embodiments, the photobioreactors rely on solar radiation for light, while others utilize man-made lights or solar collectors that channel solar radiation to the photobioreactor. In other exemplary embodiments, the water source is derived from fermenters wherein an aqueous solution is produced in the presence of sugars, cellulose, and/or other biomass. Bioreactors, that produce aqueous solutions containing either phototrophic or heterotrophic algae can be used as the water source as well.

In exemplary embodiments, the water source contains and/or is an aqueous solution containing a first population of microorganisms capable of producing glycerol.

Suitable tubular reactor water sources include those constructed from glass; or from plastics, including, but not limited to polyethylene, polypropylene, polycarbonate, acrylic, polyesters, specialty polyesters, for example, cyclohexanedimethanol modified polyesters and Tritan® from Eastman Chemical Company, and combinations thereof. The tubular photobioreactors may be constructed so that they are rigid in nature, for example, those constructed from glass, polyester, or polycarbonate.

Suitable enclosed raceway water sources include, but are not limited to, racetrack-shaped ponds that are covered to protect aqueous solution containing microorganisms capable of producing glycerol from inclement weather, and they have the ability to control the degree of agitation via a mixing device. These enclosed raceways are similar to an algal pond located in a greenhouse. This type of water source tends to be expensive, but may be preferred at latitudes greater than about 35 degrees where the winter temperatures are too low to support efficient growth of microorganisms capable of producing glycerol.

Suitable fermenter water sources include, but are not limited to, those that are commonly used in the art for the production of non-photosynthetic organisms. Fermenters are especially useful for growing genetically modified microorganisms that must be contained. Other microorganisms that are grown to relatively high concentrations in fermenters are those that require an organic carbon source, for example, sugar, fatty acid or alcohol that is not directly carbon dioxide or bicarbonate. Fermenters are a particularly advantageous water source in embodiments wherein an aqueous solution containing heterotrophic microorganisms is transferred to and utilized in the first reaction zone.

Suitable open pond water sources include, but are not limited to, those used for growing shrimp, fish, shellfish, or other types of marine and freshwater organisms, or combinations thereof. Other suitable open pond water sources include those that are used for the production of solar salt or other minerals. These open pond water sources may either be lined or unlined, although the latter provides advantages from an economic standpoint. These open ponds may be lined with plastic or bentonite or clay or other material that is impervious to the flow of water and other aqueous solutions. Pond liners constructed from various plastics including, but not limited to polyethylene, polypropylene, vinyl, and combinations thereof may be include in these water sources. Liners formed from bentonite, clay, salt, and other minerals and combinations thereof may also be useful to reduce or minimize leakage of the aqueous solution in the water source into the environment.

Enclosed photobioreactors can also be used as a water source in the methods and systems disclosed herein. These photobioreactors are transparent so that the microorganisms they contain can utilize the sunlight. These photobioreactors have also been proposed for use in the production of biofuels, nutraceuticals and specialty oils. These enclosed photobioreactors may include plastic bags, glass and plastic tubes, column photobioreactors, flat plate photobioreactors, flat panel photobioreactors, ponds in greenhouse structures, and the like. Tubular reactors were popularized by GreenFuel Technologies Corporation of Cambridge, Massachusetts for the production of biofuels, but the technology was economically unsuccessful. Plastic bag bioreactors are typified by those utilized by Algenol Biofuels of Fort Myers, Florida. Although the capital cost of constructing a bioreactor from plastic instead of steel is substantially reduced, a plastic bioreactor is still so expensive that one commercial application to date is for the production of astaxanthin, a carotenoid, which is a high-value product. Thus, the use of the enclosed photobioreactors as a water source can be included within the methods and systems disclosed herein.

Open-pond bioreactor water sources can include, but are not limited to, those generally classified as natural, intensive, and extensive. The natural open-pond bioreactors are defined as those naturally occurring ponds where the conditions are right to grow microorganisms capable of producing glycerol. These ponds may contain either fresh or saline water, and they are unmanaged in terms that they lack controlled fertilizer addition and mechanical agitation. Natural open ponds that contain microorganisms capable of producing glycerol are common along the shores of the Great Salt Lake in Utah.

Both the intensive and extensive modes of open-pond bioreactors require the controlled addition of fertilizers to the medium in order to supply the necessary nutrients, for example, phosphorus, nitrogen, iron, and trace metals, that are necessary for biomass production through photosynthesis. The primary difference between the two modes of production is mixing of the aqueous solution. Intensive open-pond bioreactors employ mechanical mixing devices while extensive open-pond bioreactors rely on happenstance mixing. Therefore, factors that affect microorganism growth can be more accurately controlled in intensive open-pond bioreactors.

Intensive open-pond bioreactors are frequently constructed by erecting a concrete block perimeter that is used to contain a plastic liner. Depth of the culture is generally controlled at about 15-30 centimeters, which has been considered to be the optimum depth for producing algal biomass. A number of configurations of these ponds have been proposed. However, the open-air raceway ponds are particularly advantageous from a commercial standpoint. Raceway ponds can employ one or more paddle wheels, pumps (such as a propeller pump or an Archimedes screw pump) and/or fluid jets to provide mixing. Chemical and biological parameters are carefully controlled, including salt and fertilizer concentrations, pH of the aqueous solution, and purity of the culture.

Extensive open-pond bioreactors have been practiced in the hot and arid regions of Australia for the production of beta-carotene. Outdoor ponds for extensive aquaculture generally are larger than those for intensive aquaculture and normally are constructed in lake beds. The open-air ponds are bounded by earthen dikes. No mechanical mixing devices are employed.

The first reaction zone (e.g., 102 in FIG. 1) can include or be a pond, such as a pond open to the atmosphere (e.g., a mechanical agitation raceway pond design, a pond without mechanical agitation, a multistep aquaculture), a pond closed to the atmosphere (e.g., a greenhouse-covered pond), a closed or semi-closed bioreactor (e.g., a fermenter), a bubble column, an airlift column, a tubular reactor, a flat panel (i.e. flat-plate) reactor, or a combination thereof. In exemplary embodiments, the first reaction zone includes or is an open pond, bioreactor, or a combination thereof.

Following the exemplary embodiment depicted in FIG. 1, during or after the addition of the aqueous solution into the first reaction zone (102), the culturing of the first population of microorganisms, that produce glycerol, in the first reaction zone occurs, thereby producing biomass material containing glycerol. Once a desirable amount of the biomass has been produced, an aqueous stream containing the biomass material is provided and inputted to the harvesting zone (104) e.g., through either a first canal, transport pipe or combination thereof (108).

As used herein, a “desirable amount” of biomass can be any amount ranging from at least about 0.05 wt % of the total weight of the aqueous solution in the first reaction zone and at most 20 wt % of the total weight of the aqueous solution in the first reaction zone.

In exemplary embodiments, the first population of microorganisms are cultured in the first reaction zone for a residence time from 1 day to 50 days, from 2 days to 50 days, from 5 days to 50 days, from 10 days to 50 days, from 20 days to 50 days or any time period falling within these ranges.

The aqueous stream can also include combinations of ions found in seawater.

The aqueous stream can contain concentrations of salts which range from trace amounts to saturating amounts. Suitable terms to describe the salinity or salt concentration of the aqueous stream range from fresh water, brackish water, salt water, brine, and saturated brine, respectively, as the salt concentration in the aqueous stream increases. The desired concentration of salt in the aqueous stream can depend on the type of microorganisms in the first population of microorganisms.

Various combinations of ions found in seawater may be included in the aqueous stream. Suitable ion combinations may be derived from one or more of the following sources including: water derived from streams, lakes, rivers, or other sources associated with fresh water; water derived from underground aquifers that may include various ion concentrations; water derived from industrial, agricultural, or municipal sources that may or may not have received treatment; or water derived from brackish sources where fresh water is combined with sea water or ocean water in various proportions; sea water or ocean water that may be derived from the various seas and oceans located around the globe; water derived from terminal lakes; an irrigation canal and/or a seawater desalination process; or combinations thereof. The combination of ions for the aqueous stream may be derived directly from these sources or may be derived by evaporating the desired amount of water from any of these sources, thereby providing a desired ion-rich solution for use in the first reaction zone and/or aqueous stream. An example of an ion combination source is disclosed e.g., in U.S. Pat. No. 6,986,323, the contents of which are incorporated herein by reference in their entirety. Other examples include the evaporation of ancient sea waters that form terminal lakes, such as the Great Salt Lake in Utah, and that form various aquifers. The combination of ions can result up to and include crystallizers wherein sodium chloride ions are precipitated.

The aqueous stream can have a salinity that is about 5 wt % or greater than 5 wt %, about 6 wt % or greater than 6 wt %, about 7 wt % or greater than 7 wt %, at least about 8 wt %, at least about 9 wt %, at least about 10 wt %, at least about 11 wt %, at least about 12 wt %, at least about 13 wt %, at least about 14 wt %, at least about 15 wt %, at least about 16 wt %, at least about 17 wt %, at least about 18 wt %, at least about 19 wt %, at least about 20 wt %, at least about 21 wt %, at least about 22 wt %, at least about 23 wt %, at least about 24 wt %, at least about 25 wt %, at least about 26 wt %, or at least about 27 wt %. In exemplary embodiments the aqueous stream is saturated with salt. In other exemplary embodiments, the aqueous stream can have a salinity that is about 5 wt % to about 27 wt % or to about saturation, from about 7 wt % to saturation, from about 10 wt % to saturation, from about 5 wt % to about 25 wt %, from about 20 wt % to saturation, from about 5 wt % to about 20 wt %, from about 10 wt % to about 20 wt %, from about 5 wt % to about 15 wt %, from about 10 wt % to about 15 wt %, or from about 5 wt % to about 10 wt %. In exemplary embodiments, the salinity of the aqueous stream, the aqueous glycerol stream and the enriched aqueous stream have about the same salinity or a salinity that does not differ more than 10 wt %, more than 5 wt %, more than 4 wt %, more than 3 wt %, more than 2 wt % or more than 1 wt % between the mentioned streams.

The aqueous stream can contain a water content of about 99.9 wt % to about 95 wt %, about 95 wt % to about 90 wt %, about 90 wt % to about 85 wt %, about 85 wt % to about 80 wt %, about 80 wt % to about 70 wt %, about 70 wt % to about 60 wt %, about 60 wt % to about 50 wt %, or about any range within 99.9 wt % to 50 wt % of the total weight of the aqueous stream. In exemplary embodiments, the aqueous stream can contain a water content greater than 50 wt % or about any range within 50 wt % to 99.9 wt %.

The first population of microorganisms can include one or more autotrophic microbes, such as algae, bacteria and/or combinations thereof. In an exemplary embodiment, the first population of microorganisms contains eukaryotic algae, prokaryotes (e.g., one or more bacteria and/or one or more archaea) or combinations thereof. Prokaryotes that may be present within the first population of microorganisms can include bacteria and archaea. The eukaryotic algae can be those commonly used in the art to create biomass materials containing glycerol.

The first population of microorganisms can also include one or more heterotrophic microbes, one or more halophilic microbes, one or more halotolerant microbes, one or more microalgae, one or more anaerobic microbes, one or more aerobic microbes, or combinations thereof.

In exemplary embodiments, the first population of microorganisms can include one or more algae from the divisions of Chlorophycophyta, Phaeophycophyta, Chrysophycophyta, Cyanophycophyta, Cryptophycophyta, Pyrrhophycophyta and/or Rhodophycophyta, which are optionally adaptable to saline water as a growth medium; or one or more microalgae species selected from, but not limited to, Amphora sp., Anabaena sp., Anabaena flos-aquae, Ankistrodesmus falcatus, Arthrospira sp., Arthrospira (Spirulina) obliquus, Arthrospira (Spirulina) platensis, Botryococcus braunii, Ceramium sp., Chaetoceros gracilis, Chlamydomonas sp., Chlamydomonas mexicana, Chlamydomonas reinhardtii, Chlorella sp., Chlorella fusca, Chlorella protothecoides, Chlorella pyrenoidosa, Chlorella stigmataphora, Chlorella vulgaris, Chlorella zofingiensis, Chlorococcum citriforme, Chlorococcum littorale, Closterium sp., Coccolithus huxleyi, Cosmarium sp., Crypthecoddinium cohnii, Cryptomonas sp., Cyclotella cryptica, Cyclotella nana, Dunaliella sp., Dunaliella bardawil, Dunaliella salina, Dunaliella tertiolecta, Dunaliella viridis, Euglena gracilis, Fragilaria sp., Fragilaria sublinearis, Gracilaria sp., Haematococcus pluvialis, Hantzschia sp., Isochrysis galbana, Microcystis sp., Monochrysis lutheri, Muriellopsis sp., Nannochloris sp., Nannochloropsis sp., Nannochloropsis salina, Navicula sp., Navicula saprophila, Neochloris oleoabundans, Neospongiococcum gelatinosum, Nitzschia laevis, Nitzschia alba, Nitzschia communis, Nitzschia paleacea, Nitzschia closterium, Nitzschia palea Nostoc commune, Nostoc flagellaforme, Pavlova gyrens, Peridinium sp., Phaeodactylum tricornutum, Pleurochrysis carterae, Porphyra sp., Porphyridium aerugineum, Porphyridium cruentum, Prymnesium sp., Prymnesium paruum, Pseudochoricystis ellipsoidea, Rhodomonas sp., Scenedesmus sp., Scenedesmus braziliensis, Scenedesmus obliquus, Scenedesmus quadricauda, Scenedesmus acutus, Scenedesmus dimorphus, Schizochytrium sp., Scytonema sp., Skeletonema costatum, Spirogyra sp., Schizochytrium limacinum, Stichococcus bacillaris, Synechococcus sp., Tetraselmis sp., Tolypothrix sp., and genetically-engineered varieties or combinations (mixtures, or mixed cultures) of these microalgal species. In exemplary embodiments, the one or more algae or microalgae present in the first population of microorganisms are selected from the group including or consisting of Dunaliella sp., Dunaliella bardawil, Dunaliella salina, Dunaliella kone, Dunaliella tertiolecta, Dunaliella parva and Dunaliella viridis, and any combination thereof. In exemplary embodiments, the algae or microalgae is Dunaliella salina, Dunaliella bardawil, Dunaliella kone, or a combination thereof.

The algae or microalgae which can be present in the first population of microorganisms can include one or more microalgal species (including diatoms, coccolithophorids and dinoflagellates) selected from, but not limited to, Amphora sp., Ankistrodesmus sp., Arthrospira (Spirulina) plantesis, Botryococcus braunii, Chlamydomonas sp., Chlamydomonas reinhardtii, Chlorella protothecoides, Chlorella sp., Closterium sp., Cosmarium sp., Crypthecoddinium cohnii, Cyclotella sp., Dunaliella salina, Dunaliella bardawil, Dunaliella tertiolecta, Haematococcus pluvialis, Hantzschia sp., Nannochloris sp., Nannochloropsis sp., Navicula sp., Neochloris oleoabundans, Nitzschia sp., Phaeodactylum tricornutum, Scenedesmus sp., Schizochytrium limacinum, Stichococcus sp., Tetraselmis suecica, and Thalassiosira pseudonana, and genetically-engineered varieties or combinations (mixtures, mixed cultures, co-cultures, or synthetic co-cultures) of these microalgal species.

The algae or microalgae which can be present in the first population of microorganisms can also include algae with flagella, cilia and/or eyespots. Flagella are a tail-like projection that protrudes from the cell body of certain algae and functions in locomotion. Cilia are an adaptation that allows independent cellular creatures, like algae, to move around in search of food. Photosensitive eyespots are found in some free-swimming unicellular algae. Photosensitive eyespots are sensitive to light. They enable the algae to move in relation to a light source. Such algae have the capability of independent motion, phototaxis, and can move towards the surface during daylight. Phototaxis is the movement of microalgae in response to light. For example, certain algae (e.g., Dunaliella) can perceive light by means of a sensitive eyespot and move to regions of higher light concentration to enhance photosynthesis.

The algae or microalgae which may be present in the first population of microorganisms also include marine algae that thrive at salt concentrations above that found in seawater. Suitable marine algae can be selected from, but are not limited to, Amphora sp. (diatom), Arthrospira sp., Arthrospira (Spirulina) obliquus, Arthrospira (Spirulina) platensis, Chlorella sp., Chlorella fusca, Chlorella protothecoides, Chlorella pyrenoidosa, Chlorella stigmataphora, Chlorella vulgaris, Chlorella zofingiensis, Dunaliella sp., Dunaliella bardawil, Dunaliella salina, Dunaliella tertiolecta, Dunaliella viridis, Isochrysis galbana, Microcystis sp., Nannochloropsis sp., Nannochloropsis salina, Navicula sp. (diatom), Navicula saprophila (diatom), Nitzschia laevis (diatom), Nitzschia alba (diatom), Nitzschia communis (diatom), Nitzschia paleacea (diatom), Nitzschia closterium (diatom), Nitzschia palea, (diatom), and genetically-engineered varieties or combinations (mixtures, mixed cultures, co-cultures, or synthetic co-cultures) of these algal species.

In exemplary embodiments, the algae in the first population of microorganisms is or includes microalgae. In other exemplary embodiments, the algae or microalgae have not been genetically modified or do not originate from genetically engineered algae or microalgae.

The first population of microorganisms can originate from at least one or a combination of plant, algae, microorganism, bacteria, or microalgae feedstock sources. Suitable algae or microalgae feedstock sources can be derived from reactors that include, but are not limited to, tubular reactors, column reactors, flat panel (i.e. flat-plate) reactors, photobioreactors, enclosed raceways, covered ponds, open raceways, open ponds, earthen ponds, ponds in greenhouses, clear plastic bags hung either indoors or outdoors, fermenters, naturally occurring bodies of water, solar salt ponds, and combinations thereof.

The biomass material present in the aqueous stream and/or first reaction zone can include or be a plant biomass, a microbial biomass, an algal biomass or any combination thereof.

All of the possible plant and/or microbe species which can be included in the first population of microorganisms can also be included within the biomass material in the aqueous stream.

The biomass material can also include or contain some or all of the natural products produced by the first population of microorganisms.

The biomass material content in the aqueous stream can be as low as about 0.01 wt % or about 0.05 wt %, but it can be greater than about 0.5 wt % or greater than 1 wt %. In exemplary embodiments the maximum biomass material content in the aqueous stream is less than about 20 wt %. Any content greater than 20 wt % may slow the flow of the aqueous stream through the systems described herein, thereby leading to longer purification times and an increased chance in a blockage forming within the systems. In exemplary embodiments, the maximum content of biomass material in the aqueous stream is less than about 10 wt %.

The biomass material can include or be a conditioned biomass. As used herein “a conditioned biomass” refers to a biomass material that has been treated with one or more conditioning processes before separating from the aqueous stream in the harvesting zone. Suitable conditioning processes can include, but are not limited to, subjecting the biomass material to fracking methods, a pressure drop across an orifice, vibratory mill grinding system, osmotic shock, and other methods previously disclosed in the art.

The biomass material can include useful or valuable components. These components can be produced intracellularly and/or extracellularly from microbial cell populations within the biomass material. These microbial cells containing the valuable components can be recovered from the first reaction zone and transferred to the harvesting zone.

The biomass material can contain glycerol and other products (e.g., natural products) formed from or by the first population of microorganisms.

The expression “natural products” refers to products which are naturally produced or found within an environment of a living organism. Natural products can include those which are hydrophobic, hydrophilic or amphipathic.

In exemplary embodiments, the biomass can contain natural products produced by a plant, a microbe, an algae or microalgae species, these products including lipids, algal lipids, carotenoids, fatty acids, algal fatty acids, triacylglycerols, diacylglycerols, monoacylglycerols, oils, algal oils, chlorophyll, glycerol, phospholipids, carbohydrates, fibers, proteins or combinations thereof.

The biomass material can contain glycerol e.g., up to about 85 wt %, 10 wt % to 27 wt % or 16 wt % to 26 wt % of the total biomass material weight.

The aqueous stream containing the microbial biomass can contain a degree of salinity from about 5 wt % to about 27 wt % salt in the aqueous stream. In exemplary embodiments, the aqueous stream can be saturated in salt, which, for example, for sodium chloride, the saturating degree of salinity is about 27 wt % at 25 °C.

The expression “salinity” refers to the total amount of dissolved salts that can be present within the biomass material and/or the medium in which the biomass material resides, such as an aqueous stream. Salts which can be dissolved and found in the biomass material and/or its medium include, but are not limited to, those found in natural waters such as sodium chloride, magnesium chloride, calcium and magnesium sulfates, bicarbonates, and carbonates. In more general terms, salinity is indicated by the water source, such as a freshwater, a brackish water, a saline water, and a brine. Ranges of salinity are associated with these general terms and these ranges are defined as <0.05 wt % for freshwater, 0.05-3 wt % for brackish water, 3-5 wt % for saline water, and >5 wt % for a brine.

As used herein, wt % refers to a dry mass of a component in a solution in grams divided by 100 grams of the solution.

In exemplary embodiments, inputting the aqueous stream with the biomass material containing glycerol to the harvesting zone can include feeding the aqueous stream through a first transport line that is in communication with the first reaction zone and the harvesting zone containing the harvester. The first transport line can either be in direct or indirect communication with the harvesting zone and/or the first reaction zone.

In exemplary embodiments, the inputting of the aqueous stream with the biomass material containing glycerol to the harvesting zone can include feeding the aqueous stream through a hydraulic pump located on the first transport line.

The harvesting zone includes a harvester. In exemplary embodiments, the harvester contains or is an adsorptive bubble separation unit. The adsorptive bubble separation unit can utilize a flotation aid, a frother, a collector, and/or an activator, or any combination thereof. In other exemplary embodiments, the harvester includes or is a filter, a deep bed filter, a belt press, a screw press, a centrifuge, an adsorber, a sedimentation unit, a mechanical flotation unit, a dissolved gas flotation unit, a froth flotation unit, a flocculation unit, or any combination thereof.

Collectors selectively render one or more molecules or natural products in the biomass material hydrophobic, thereby assisting in the process of collecting biomass material on gas bubbles. Activators aid in the adsorption of the collector to certain molecules or natural products in the biomass material, thereby increasing the number of those molecules or products which become hydrophobic. Depressors inhibit the adsorption of the collector to undesirable molecules or natural products in the biomass material, thereby decreasing the number of those molecules or products which become hydrophobic. Also, frothing agents and frothers may be added to the harvesting zone to assist in the formation of a stable froth containing hydrophobic molecules or natural products from the biomass material on the surface of a liquid.

The harvesting zone may include at least one or more harvesting units or equipment for the centrifugation, flocculation, sedimentation, and/or filtration of the biomass material. Such equipment or units are described e.g., in U.S. Pat. No. 5,776,349; the contents of which are incorporated herein by reference in their entirety. These harvesting units or equipment can also, optionally, be used in combination with adsorptive bubble separation units in the harvesting zone.

Any sedimentation unit or equipment known in the art may be present within the harvesting zone. For example, sedimentation units that can add alum to the biomass material and/or not agitate the biomass material when in an aqueous stream or media can be included within the harvesting zone. Other possible sedimentation units that can be present in the harvesting zone are those that can add ferric chloride and/or polymers or ions that cause flocculation of the biomass material. Cyclone sedimentation units can also be included within the harvesting zone. Skimming units can also be included within the harvesting zone.

Deep bed filtration equipment may also be present in the harvesting zone. This equipment can be used to pre-concentrate the biomass material prior to an adsorptive bubble separation process. Deep bed filtration relies upon a bed of granular media, usually sand, through which an aqueous stream containing the biomass material flows downward under gravity. The biomass material can be deposited in the pores of the granular media and in the interstitial spaces between the grains of media. Deep bed filtration should not be confused with straining filtration. Straining takes place on the surface of a mesh or fabric and is only suitable to pre-concentrate an aqueous stream with natural products that will not blind the filtration equipment.

The harvesting zone can include equipment or units that can rupture cells contained in the biomass material. The biomass material includes cellular material that contains natural products, such as glycerol. Therefore, rupturing the cell wall and/or cell membrane of the cellular material can release natural products that can be later purified. Cell rupturing can be achieved by a number of methods which include, but are not limited to, chemical, physical or mechanical methods. Chemical methods can include enzymatic digestion, detergent solubilization, lipid dissolution with a solvent, and alkali treatment (lipid saponification). Physical methods can include osmotic shock, decompression, sonication, heat treatment, and freeze-thawing. Mechanical methods can include grinding, high shear homogenization, passing the feedstock stream across a pressure drop, and pressure extrusion.

Other cell disruption processes which can be used include pumping the feedstock stream at high pressures through a restricted orifice valve. An equipment which can perform this disruption method is, as an example, the MICROFLUIDIZER™ cell disruption equipment of Microfluidics, Newton, MA, US, which utilizes pressures of about 5,000 to 40,000 psig (345-2760 bar).

A mill, such as a vibratory mill, can also be present in the harvesting zone and used to rupture cellular material in the biomass material.

In exemplary embodiments wherein the biomass material contains algae or microalgae components, the harvesting zone can include equipment that is capable of fracking the biomass material. The partial rupturing of algae is referred to as fracking. Fracking may take place in any device known in the art in which algae or microalgae may be partially ruptured including, but not limited to, a vibratory mill, a French press, a pump, an agitated vessel, or combinations thereof. In some embodiments, fracked algae are advantageous to use over completely ruptured algae due to the difference in size of the resulting particles. Particles resulting from fracking algae are larger than the particles resulting from the complete rupturing of algae and thus adsorptive bubble separation processes could be more effective when larger particles are present. Fracking the algae or microalgae can produce fracked cells possessing hydrophobic components while still retaining a significant portion of the intracellular material within the cellular membrane. This can result in increased recovery of the intracellular material and the natural products contained within the cells.

Following the exemplary embodiment depicted in FIG. 1, once the aqueous stream including the biomass and glycerol reaches the harvesting zone (104), the biomass undergoes additional processing and at least part of the biomass material is effectively separated from the aqueous stream containing the glycerol. The separated algal biomass is then transported out of the harvester (104) e.g., via a first recovery line (110), thereby providing an aqueous glycerol stream containing glycerol. The aqueous stream containing glycerol is removed from the harvesting zone (104) and supplied to a second reaction zone (106) containing a second population of microorganisms. The aqueous stream containing glycerol can be supplied to the second reaction zone (106) from the harvesting zone (104) via, e.g., a second canal, a second transport pipe or a combination thereof (112).

In exemplary embodiments, the biomass material contains useful or valuable components and is recovered and/or valorized (i.e., without separating the valuable components from the biomass material) from the first reaction zone and/or harvesting zone.

The separating of the biomass material from the glycerol in the harvesting zone can include subjecting the aqueous stream including the biomass material to conditioning processes that can include, but are not limited to, fracking, belt pressing, screw pressing; and/or concentration processes that include, but are not limited to, adsorptive bubble separation, filtration, deep bed filtration, centrifugation, adsorption, sedimentation, mechanical flotation, froth flotation, flocculation and combinations thereof.

In exemplary embodiments, the separating of the biomass material from the glycerol in the harvesting zone includes rupturing the biomass material to release the glycerol.

In exemplary embodiments, once the biomass material has been effectively separated from the aqueous stream containing glycerol in the harvesting zone, the biomass material can be effectively removed from the harvester in the harvesting zone by recovering the biomass material through a first recovery line in communication with the harvester. In one embodiment, the first recovery line is formed from a material of construction that can withstand the corrosive nature of salt. Suitable materials of construction for the first recovery line include, but are not limited to stainless steel, hastelloy, polyvinyl chloride, high-density polyethylene, glass, fiberglass reinforced plastic, and combinations thereof. In one embodiment, the biomass material flows through the first recovery line by gravity. In yet another embodiment, the biomass material flows through the first recovery line by the force of a pump, e.g., a pump that delivers a lower amount of shear forces relative to that generated by a centrifugal pump.

The aqueous glycerol stream can have a dissolved glycerol concentration from 0.1 to 20000 ppm, from 1.0 to 20000 ppm, from 10 to 20000 ppm, from 50 to 20000 ppm, from 100 to 20000 ppm, from 500 to 20000 ppm, from 1000 to 20000 ppm or any concentration falling within the above ranges.

The aqueous glycerol stream can have any salinity mentioned in the present disclosure for the aqueous stream containing biomass material and glycerol. For example, a salinity can be from about 5 wt % to about 27 wt %, from about 5 wt % to about 25 wt %, from 10 wt % to about 27 wt %, from 15 wt % to about 27 wt %, from 20 wt % to about 27 wt %, from 25 wt % to about 27 wt % or any salinity falling within the above ranges. In exemplary embodiments, the aqueous glycerol stream can have a salinity similar to the aqueous stream.

The aqueous glycerol stream can have a water content that falls within the possible water content ranges of the aqueous stream.

In exemplary embodiments, the aqueous glycerol stream is contacted with an oxidizing agent and/or exposed to ultraviolet radiation optionally before the aqueous glycerol stream enters the second reaction zone. The aqueous glycerol stream can be contacted with an oxidizing agent including, but not limited to, ozone, hydrogen peroxide, chlorine, hypochlorite, chlorite or combinations thereof. For example, the aqueous glycerol stream may be contacted with an oxidizing agent within and/or after the harvesting zone. In an exemplary embodiment, the aqueous glycerol stream may be exposed to ultraviolet radiation within and/or after the harvesting zone. One or more oxidizing agents, ultraviolet radiation, or a combination thereof can be used to partly or fully oxidize residual microbial or algal biomass and microbial or algal components of the aqueous glycerol stream thereby converting them into CO₂ and/or to control the level of other organisms in the stream.

Supplying the aqueous glycerol stream to the second reaction zone containing a second population of microorganisms that metabolize glycerol can include feeding the aqueous glycerol stream into a second transport line in communication with the harvesting zone and the second reaction zone. The second transport line can either be in direct or indirect communication with the harvesting zone and/or the second reaction zone.

In exemplary embodiments, the supplying of the aqueous glycerol stream to the second reaction zone can include feeding the aqueous glycerol stream through a hydraulic pump located on the second transport line.

The second reaction zone may include or be a polishing unit (e.g., a biological polishing unit) that is either a pond open to the atmosphere, a mechanically agitated pond (e.g., raceway pond design), a pond without mechanical agitation, a pond closed to the atmosphere (e.g., a greenhouse-covered pond), a bio-film based reactor (e.g., a biofilter, a trickle bed reactor or fluidized bed bioreactor), a moving bed biofilm reactor, a closed or semi-closed bioreactor (e.g., a fermenter, a pipeline/vessel containing a UV-radiation source, a pipeline/vessel containing an oxidizing agent, a protein skimmer, a fixed-film bioreactor), or any combination thereof. In exemplary embodiments, the second reaction zone includes or is a biological polishing system, the polishing system being either an open pond, bioreactor, or combination thereof.

The second population of microorganisms inhabiting the second reaction zone can include any one or combination of microorganisms that can be included within the first reaction zone.

In exemplary embodiments, the second population of microorganisms includes or are heterotrophic and/or mixotrophic microorganisms that can utilize glycerol to produce useful biological materials, such as CO₂, lipids, proteins and other molecules such as macromolecules.

In other exemplary embodiments, the second population of microorganisms include halophilic and/or halotolerant microorganisms that are optionally capable of growing under saturating salt conditions and/or autotrophic microorganisms.

In one exemplary embodiment, the second population of microorganisms includes one or more anaerobic microbes and/or one or more aerobic microbes.

In one exemplary embodiment the first population and/or the second population of microorganisms include(s) one or more bacteria and/or archaea selected from Halobacterium, Halomonas, Haloquadratum, Halosimplex, Haloferax, Haloarcula, Halorubrum, Natrialba, Natronobacterium, Natronococcus, Salinibacter, Spiribacter, and any combination thereof.

In some exemplary embodiments, the first population of microorganisms and/or the second population of microorganisms contain eukaryotic algae cells and prokaryotic cells. Such prokaryotic cells may include bacteria and/or archaea cells. The ratio of eukaryotic algae cells (cells/ml) to prokaryote cells (cells/ml) can be higher in the first population than in the second population and/or the ratio of prokaryote cells (cells/ml) to eukaryotic algae cells (cells/ml) can be higher in the second population of microorganisms than in the first population of microorganisms.

The separation of at least part of the biomass material from the aqueous stream in the harvesting zone produces an aqueous glycerol stream containing a reduced amount of biomass material. In exemplary embodiments, the aqueous glycerol stream flows through a second transportation pipe or canal by gravity from the harvesting zone into the second reaction zone.

Following the exemplary embodiment depicted in FIG. 1, once the aqueous glycerol stream containing glycerol enters into the second reaction zone (106), the second population of microorganisms inhabiting the second reaction zone (106) are cultivated with the aqueous glycerol stream. During cultivation, the second population of microorganisms metabolize the glycerol in the aqueous glycerol stream into useful CO₂, lipids, proteins or other molecules such as macromolecules. These useful glycerol metabolites are recovered from the second reaction zone (106) e.g., by a second recovery line (114), thereby providing a depleted aqueous stream. The depleted aqueous stream, now having a significantly reduced amount of glycerol, is transported from the second reaction zone (106) back to the first reaction zone (102) e.g., via a third canal, third transport pipe or a combination thereof (116).

In exemplary embodiments, the aqueous glycerol stream is cultivated in the second reaction zone for a residence time from 1 day to 50 days, from 2 days to 50 days, from 5 days to 50 days, from 10 days to 50 days, from 20 days to 50 days or any time period falling within these ranges. In exemplary embodiments where the second reaction zone is a bioreactor, for example a biofilter or a moving bed biofilm reactor, the residence time may range from several days to less than an hour.

In exemplary embodiments, the ratio of eukaryotic algae cells in the first population of microorganisms to eukaryotic algae cells in the second population of microorganisms is more than 1:1, more than 10:1, more than 100:1, more than 1000:1, more than 5000:1, or more than 10,000:1 and/or the ratio of the prokaryote cells in the first population of microorganisms to the prokaryote cells in the second population of microorganisms is less than 1:1, less than 1:2, less than 1:5, less than 1:10, less than 1:50, less than 1:100, or less than 1:1000.

In exemplary embodiments, the ratio of eukaryotic algae cells/ml in the first population of microorganisms to eukaryotic algae cells/ml in the second population of microorganisms is more than 1, and/or a ratio of prokaryote cells/ml in the first population of microorganisms to prokaryote cells/ml in the second population of microorganisms is less than 1 or less than 1:1.

In exemplary embodiments, the ratio of eukaryotic algae cells/ml to prokaryote cells/ml is higher in the first population of microorganisms than in the second population of microorganisms; the first population of microorganisms contains at least one or more of eukaryotic alga, prokaryote, and/or combinations thereof; the second population of microorganisms contains at least one or more of eukaryotic alga, prokaryote and/or combinations thereof; the second population of microorganisms contains more prokaryote cells/ml than the first population of microorganisms; and/or the first population of microorganisms contains more eukaryotic algae cells/ml than the second population of microorganisms.

In exemplary embodiments, the method for removing glycerol from the aqueous system can include recovering an enriched aqueous stream in a second recovery line that is in communication with the second reaction zone. The second recovery line can be configured to recover glycerol metabolites and useful degradation products from the enriched aqueous stream. In exemplary embodiments, the second recovery line is formed from a material of construction that can withstand the corrosive nature of salt. Suitable materials of construction for the second recovery line include, but are not limited to stainless steel, hastelloy, polyvinyl chloride, high-density polyethylene, glass, fiberglass reinforced plastic, and combinations thereof.

The methods for removing glycerol from an aquaculture system described herein can include recovering the glycerol metabolites from the enriched aqueous stream, thereby providing a depleted aqueous stream. The glycerol metabolites that can be recovered from the enriched aqueous stream include, but are not limited to, water, carbon dioxide, biomass, proteins, lipids, carotenoids, polymers, alcohols, acids, diols and combinations thereof.

In exemplary embodiments, the depleted aqueous stream possesses a lower amount of glycerol and/or glycerol metabolites than the enriched aqueous stream.

The enriched aqueous stream and/or the depleted aqueous stream can also possess a salinity that falls within the salinity ranges for the aqueous stream containing biomass and glycerol and/or the aqueous glycerol stream. In exemplary embodiments, the enriched aqueous stream has a salinity between about 5 wt % and saturation.

In exemplary embodiments, the enriched aqueous stream and/or the depleted aqueous stream possess a ratio of eukaryotic algae cells to prokaryote cells that is less than 1:1, less than 1:2, less than 1:5, less than 1:10, less than 1:50, less than 1:100, less than 1:500, or less than 1:1000. In exemplary embodiments, the enriched aqueous stream and/or the depleted aqueous stream possess a ratio of eukaryotic algae cells to prokaryote cells less than 1.

In exemplary embodiments, the enriched aqueous stream and/or the depleted aqueous stream possess low levels of carbon, phosphorus and/or nitrogen. The amount of total organic carbon present in these streams can range from 10 to 400 mg/L, e.g., from 10 to 100 mg/L. The amount of chemical oxygen demand present in these streams can range from 30 to 900 mg/L, e.g., from 30 to 300 mg/L. The amount of phosphorus present in these streams can range from 0.3 to 300 mg/L, e.g., from 0.3 to 100 mg/L, from 0.3 to 50 mg/L or from 0.3 to 10 mg/L. The amount of nitrogen present in these streams can range from 0.5 to 400 mg/L, e.g., from 0.5 to 100 mg/L, from 0.5 to 50 mg/L or from 0.5 to 10 mg/L. In exemplary embodiments, the enriched aqueous stream and/or the depleted aqueous stream contain amounts of carbon, nitrogen and/or phosphorus that are a magnitude higher or lower than the amounts described above. In exemplary embodiments, the depleted aqueous stream contains the amount of carbon, nitrogen and/or phosphorus that allow the stream to be discharged to a body of water, including but not limited to an ocean, to a sea, to a lake, to a river, to a shrimp pond, to an irrigation canal, to a salt pond, or combinations thereof.

In exemplary embodiments, the depleted aqueous stream contains reduced levels of organic carbon, phosphorus and/or nitrogen relative to the enriched aqueous stream.

In exemplary embodiments, the methods for removing glycerol from an aquaculture system described herein include recycling at least part of the enriched aqueous stream and/or depleted aqueous stream back to the first reaction zone. In some of these embodiments, the enriched aqueous stream and/or depleted aqueous stream contain 10 to 50 mg/L of nitrogen and/or 0.3 to 30 mg/L, or 1 to 2 mg/L of phosphorus.

In other exemplary embodiments, the methods for removing glycerol from an aquaculture system described herein include purging the enriched aqueous stream and/or depleted aqueous stream into an ocean, sea, river or stream. In some of these embodiments, the enriched aqueous stream and/or depleted aqueous stream contain 0.5 to 10 mg/L, or 10 to 15 mg/L of nitrogen; 1 to 2 mg/L, or at most 300 mg/L of phosphorus; and/or at most 150 mg/L (such as at most 100 mg/L, 50 mg/L or 25 mg/L) of chemical oxygen demand.

In exemplary embodiments, the methods for removing glycerol from an aquaculture system described herein include sending the enriched aqueous stream and/or depleted aqueous stream to a solar evaporation salt crystallizer pond wherein the aqueous media of the enriched aqueous stream and/or depleted aqueous stream is evaporated to produce a salt product. In some of these embodiments, the enriched aqueous stream and/or depleted aqueous stream contain 10 to 30 mg/L of nitrogen and/or 0.3 to 10 mg/L, or 1 to 2 mg/L of phosphorus.

As described herein, the total organic carbon, chemical oxygen demand, nitrogen and phosphorus are values measured from unfiltered samples. That means that for example a sample of the enriched aqueous stream or depleted aqueous stream for one or more measurements can include microorganisms or parts thereof.

In other embodiments, the methods for removing glycerol described herein include injecting an effluent stream from the second reaction zone into the first reaction zone, thereby providing a continuous glycerol removal method. In exemplary embodiments, the methods for removing glycerol include recovering the glycerol metabolites from the enriched aqueous stream and providing an effluent stream that is injected into the first reaction zone, thereby providing a continuous reactor system.

In exemplary embodiments, the aquaculture system is a continuous flow reactor system.

As described herein, a “continuous flow” method or system refers to a method or system that can operate at least in a semi-continuous flow and/or operation. That is, a method or system which has the different aqueous streams flowing in and out of the various system zones continuously or periodically.

In exemplary embodiments, the methods described herein include recycling the enriched aqueous stream and/or depleted aqueous stream into the first reaction zone by flowing the enriched aqueous stream and/or depleted aqueous stream through a third transport line in communication with the second reaction zone and the first reaction zone. The third transport line can be in either direct or indirect communication with the second reaction zone and the first reaction zone.

In some embodiments, the methods for removing glycerol described herein are performed in batch-wise operations.

As used herein “removing glycerol” refers to removal or reduction of any amount of glycerol (e.g., dissolved glycerol) or complete removal of glycerol (e.g., dissolved glycerol) from any of the streams produced by the methods and or formed in the systems described herein, for example, the aqueous glycerol stream. In exemplary embodiments, the removal of glycerol occurs in the second reaction zone. In an exemplary embodiment, the dissolved glycerol concentration of the enriched aqueous stream ranges from about 1% to about 90%, about 1% to about 80%, about 1% to about 70%, about 1% to about 60%, about 1% to about 50%, about 1% to about 40%, about 1% to about 30%, about 1% to about 20%, about 1% to about 10%, or about 1% to about 5% of the dissolved glycerol concentration in the aqueous glycerol stream.

Accordingly, another aspect of the present disclosure relates to compositions containing any of the glycerol metabolites and/or the valuable components (e.g., components derived from the biomass material, the metabolites and/or components being separated from the aqueous streams) that can be recovered from at least one or more of the enriched aqueous stream, the aqueous glycerol stream, the depleted aqueous stream and/or the separated biomass stream formed from the methods and systems disclosed herein.

In exemplary embodiments, the composition includes glycerol metabolites recovered from at least one or more of the enriched aqueous stream, the aqueous glycerol stream, the depleted aqueous stream and/or the separated biomass stream produced from the methods and/or system disclosed herein. The glycerol metabolites can be present in the composition in a concentration that ranges from about 1 wt % to about 90 wt %, about 1 wt % to about 80 wt %, about 1 wt % to about 70 wt %, about 1 wt % to about 60 wt %, about 1 wt % to about 50 wt %, about 1 wt % to about 40 wt %, about 1 wt % to about 30 wt %, about 1 wt % to about 20 wt %, about 1 wt % to about 10 wt %, or about 1 wt % to about 5 wt % of a total weight of the composition.

The enriched aqueous stream and/or the separated biomass material can include at least one hydrophobic natural product. Possible hydrophobic natural products can include, but are not limited to, one or more lipids, algal lipids, carotenoids, fatty acids, algal fatty acids, triacylglycerols, diacylglycerols, monoacylglycerols, oils, algal oils and combinations thereof.

The carotenoids may include beta-carotene, alpha-carotene, lutein, zeaxanthin, beta-cryptoxanthin, astaxanthin, phytoene, phytofluene, lycopene, and/or combinations thereof.

Any of a variety of products can be made from the biomass material and/or the glycerol metabolites recovered from any of the streams disclosed herein including, but not limited to, biofuels, nutraceuticals, cosmaceuticals, wastewater treatment processes, spa products, animal feeds, human food, soil builders, chemical intermediates, specialty lipids, solar salt, and combinations thereof.

Biofuels that may be produced from high temperature processing of the biomass material and/or the glycerol metabolites include, but are not limited to, biodiesel, green diesel, renewable diesel, sustainable aviation fuel, jet fuel, marine fuel, methane, hydrogen, alcohols, and dried algal biomass. Algal biodiesel is produced via any transesterification process known in the art, including those which utilize two immiscible liquid phases, and those that utilize a solid acid catalyst. For example, sustainable aviation fuel, renewable diesel or green diesel may be produced by hydrogenation, cracking, or a combination thereof of the algal oil or any derivative thereof in order to produce hydrocarbons that can be used directly in the existing diesel distribution system. Methane and/or hydrogen may be produced from the biomass material by any anaerobic process known in the art. Fermentation of the biomass material by any process known in the art may be used to produce methanol, ethanol, butanol, n-butanol, i-butanol, other alcohols, and combinations thereof. The biomass material may be torrified for the production of a soil builder or for use in combination with coal for power or steam generation. The biomass material may be processed via hydrothermal liquefaction or hydrothermal carbonization to produce bio-based oil (also referred to as biocrude or biocrude oil), chemicals and char. The biomass material may be dried and then gasified, pyrolyzed, or combusted either by itself or in combination with coal, biomass or municipal solid waste.

The biomass material and/or the glycerol metabolites may be extracted to recover lipids that can be used as an animal feed ingredient, renewable plastics, renewable polymers, renewable chemicals, nutraceutical, cosmaceutical, soap or components of a soap or detergent composition, and cosmetic ingredients, including, but not limited to carotenoids, omega fatty acids, and other lipids. For the production of solar salt, the biomass material may be removed from solar salt works in order to improve the salt quality. The quality of sodium chloride, sodium carbonate, and other salts can be improved by this method. Biomass material and/or the glycerol metabolites stabilized with a high temperature treatment process may also be used in animal nutrition, especially for shrimp and fish aquaculture diets. The biomass material and/or the glycerol metabolites may also be treated with a high temperature process to stabilize them against degradation during transportation. Alternatively, high temperature processing could be used to stabilize the biomass material and/or the glycerol metabolites prior to their storage for carbon sequestration purposes. The biomass material and/or the glycerol metabolites may be used to derive valuable chemical intermediates such as fatty acids for the production of polyurethanes.

Suitable animal feeds include, but are not limited to, feeds for shrimp, fish, shellfish, brine shrimp, chickens, poultry, cows, ducks, dogs, pigs, sheep, goats, and combinations thereof. The animal feeds may require the biomass material and/or the glycerol metabolites to be dried, but in some cases, for example for use in shrimp and fish aquaculture diets, complete drying may not be necessary as long as stability is sufficient.

Suitable dietary supplements include, but are not limited to alpha carotene, betacarotene, lutein, zeaxanthin, cryptoxanthin, phytoene, phytofluene, and the various cis-and trans-isomers and the various alpha, beta, gamma, delta isomers of the various carotenoids, and combinations thereof. Suitable dietary supplements also include various unsaturated fatty acids as well as protein meal, protein concentrates, protein isolates biomass powder and/or algae biomass powder.

Suitable methods of carbon storage include, but are not limited to, burying the biomass material and/or the glycerol metabolites, sinking them, hydrothermal carbonization, pyrolysis to make biochar, torrefaction and using them as a soil builder or in soil amendment, and/or combinations thereof.

Suitable methods for water and wastewater treatment include, but are not limited to, removal of BOD (biological oxygen demand), COD (chemical oxygen demand) and/or TOC (total organic carbon) from a water stream. This may be useful for municipal, agricultural and industrial wastewater treatment processes, shrimp or fish wastewater treatment, and it may be important for the treatment of brines being used for the production of sodium chloride salt and other salts via evaporation.

Suitable methods to process the biomass material and/or the glycerol metabolites into useful compounds include, but are not limited to, torrefaction, gasification, pyrolysis, liquefaction, hydrothermal liquefaction, fermentation, anaerobic digestion, drying, combustion, burial, and combinations thereof. Suitable applications of the torrefied biomass material and/or the glycerol metabolites include, but are not limited to, a soil builder and a material to be combined with coal, wood, or other combustible material for power generation, and biochar. Suitable applications of gasified biomass material and/or glycerol metabolites includes, but are not limited to, the production of the entire suite of products that can be produced via syngas chemistry, as described by the Gasification Technologies Council. Suitable products from syngas include, but are not limited to, chemicals, fertilizers, power generation, substitute natural gas, hydrogen, and transportation fuels. Suitable chemicals include, but are not limited to, hydrogen, carbon monoxide, methanol, dimethyl ether, acetic acid, propionic acid, butyric acid, acetic anhydride, methyl acetate, ethylene, propylene, olefins, and combinations thereof. Suitable fertilizers that can be produced from the syngas include, but are not limited to ammonia, ammonium nitrate, urea, and others known in the art. Suitable substitute natural gas can be generated from the syngas produced by gasifying the biomass material and/or the glycerol metabolites, and this includes methane. Suitable liquid fuels include gasoline, diesel fuel, jet fuels, bunker fuels, and combinations thereof. All of the chemicals that are produced by Eastman Chemicals and by Sasol via their gasification processes may also be produced by the gasification of the biomass material and/or the glycerol metabolites. Products produced by the utilization of syngas may also be produced by gasification of the biomass material and/or the glycerol metabolites. Illustrative processes are described in U.S. Pat. No. 6,310,260, the contents of which are incorporated herein by reference in their entirety, include, for example, hydroformylation, hydroacylation (intramolecular and intermolecular), hydrocyanation, hydroamidation, hydroesterification, aminolysis, alcoholysis, hydrocarbonylation, reductive hydroformylation, hydrogenation, olefin oligomerization, hydroxycarbonylation, carbonylation, olefin isomerization, transfer hydrogenation and the like. Other processes involve the reaction of organic compounds with carbon monoxide, or with carbon monoxide and a third reactant, e.g., hydrogen, or with hydrogen cyanide, in the presence of a catalytic amount of a metal-organophosphorus ligand complex catalyst. More advantageous processes include hydroformylation, hydrocyanation, hydrocarbonylation, hydroxycarbonylation and carbonylation.

Another aspect of the present disclosure relates to an aquaculture system, wherein glycerol is removed from the system, that includes: (i) a first reaction zone including a first population of microorganisms that produce glycerol (e.g., within a biomass material), (ii) a harvesting zone in communication with the first reaction zone, the harvesting zone including a harvester and (iii) a second reaction zone in communication with the harvesting zone, the second reaction zone including a second population of microorganisms that metabolize glycerol.

The first reaction zone can either be in direct or indirect communication with the harvesting zone including the harvester (e.g., 104 in FIG. 1).

The harvesting zone can either be in direct or indirect communication with the second reaction zone (e.g., 106 in FIG. 1). In exemplary embodiments, the second reaction zone is at least partly included within the harvesting zone.

The second reaction zone can be in direct or indirect communication with the first reaction zone. In exemplary embodiments, the second reaction zone is in communication with the first reaction zone to provide a continuous flow reactor system.

In exemplary embodiments, the systems described herein can include a first and/or second recovery line. The first recovery line (e.g., 110 in FIG. 1) can be in communication with the harvesting zone and can mediate the removal of the biomass material from an aqueous stream containing glycerol and, optionally, salt. Indeed, the first recovery line can be configured to recover microbial biomass material from the harvesting zone. The second recovery line can be in communication with the second reaction zone and can mediate the removal of glycerol degradation products or metabolites from an aqueous stream optionally including salt (e.g., 114 in FIG. 1). The second recovery line can be configured to recover glycerol degradation products or metabolites from the second reaction zone. The second recovery line can optionally be in communication with solar evaporation salt production facility and/or naturally occurring bodies of water.

In other exemplary embodiments, the systems described herein can include a first, a second, and/or a third transport line (e.g., 108, 112 and 116 in FIG. 1). The first transport line (108) can be in communication with the first reaction zone and the harvesting zone. The first transport line can mediate the exchange of material from the first reaction zone to the harvesting zone. The second transport line (112) can be in communication with the harvesting zone and the second reaction zone. The second transport line can mediate the exchange of material from the harvesting zone to the second reaction zone. The third transport line (116) can be in communication with the second reaction zone and the first reaction zone. The third transport line can mediate the exchange of material from the second reaction zone to the first reaction zone.

One or more recovery lines and/or one or more transport lines may either be canals, canals lined with waterproof materials, pipes, tubes or any combinations thereof. In exemplary embodiments, the communication between the harvesting zone and first reaction zone and/or the communication between the harvesting zone and second reaction zone is provided by a pipe, tube and/or canal. One or more recovery lines and/or one or more transport lines may be formed from plastic, concrete, clay, clay containing earthen material, and/or any material resistant to salt corrosion. In some embodiments, the transport lines are canals possessing a cover, which is optionally lined with corrosion resistant materials, such as materials resistant to salt damage and/or wear.

The liquid flow rate through one or more of the recovery lines and/or one or more of the transport lines that can be canals, canals lined with waterproof materials, pipes, tubes, or combinations thereof may be controlled by a flow control structure. Suitable flow control structures include weirs, gates, control valves, eductors, siphons and other methods known in the art, and combinations thereof.

In other exemplary embodiments, the systems described herein also include a water source (e.g., 100 in FIG. 1) and a water feed line (e.g., 118 in FIG. 1). The water feed line is in communication with the water source and the first reaction zone. The water feed line can mediate the exchange of water from the water source to the first reaction zone.

In exemplary embodiments, the systems described herein may include a hydraulic pump. The hydraulic pump can be placed on any of the transport lines described herein and/or the water feed line.

In exemplary embodiments, the system described herein may include an aquaculture pretreatment unit configured for conditioning an aqueous medium with at least algal nutrients to obtain an aquaculture medium, wherein the aquaculture pretreatment unit is arranged to feed the aquaculture medium to the one or more first reaction zones optionally by gravity feed; and/or one or more ponds for crystallizing salt, optionally in communication with the second reaction zone.

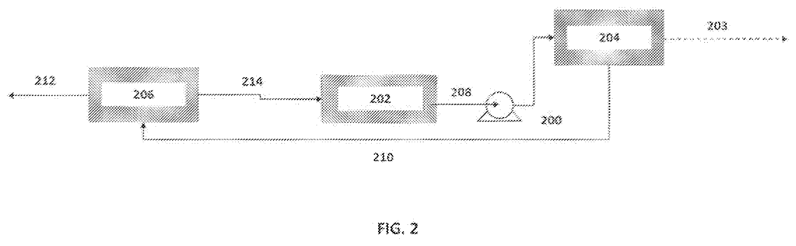

FIG. 2 depicts an exemplary aquaculture system, wherein glycerol is removed from the system, containing a hydraulic pump (200) located on a first transport line (208) in communication with a first reaction zone (202) and a harvesting zone (204). In this system, biomass material containing glycerol is suspended in an aqueous stream and is transported from the first reaction zone (202) along the first transport line (208). The aqueous stream containing the biomass material and glycerol is then passed through a hydraulic pump (200) and pumped along the first transport line (208) to a harvesting zone (204).

Once the aqueous stream including the biomass material and glycerol reaches the harvesting zone (204), the biomass is effectively separated from the aqueous stream containing glycerol, thereby creating a separated biomass stream, and removed from the harvesting zone for optional processing through a first recovery line (203) in communication with the harvesting zone (204).

The aqueous glycerol stream containing the glycerol is removed from the harvesting zone (204) and transported to a second reaction zone (206) along a second transport line (210).

Once the aqueous glycerol stream containing the glycerol enters into the second reaction zone (206), a second population of microorganisms inhabiting the second reaction zone (206) degrade the glycerol in the aqueous glycerol stream or metabolize the glycerol in the aqueous glycerol stream into useful metabolites including, but not limited to CO₂, lipids, proteins or other molecules. These useful glycerol metabolites can be either recovered from the second reaction zone (206) via a second recovery line (212) or recycled to the first reaction zone (202) via a third transport line (214) to serve as nutrients to the organisms in the first reaction zone (202).

Additionally, a depleted aqueous stream having a significantly reduced amount of glycerol can be transported from the second reaction zone (206) back to the first reaction zone (202) via the third transport line (214).

In exemplary embodiments wherein the first reaction zone and second reaction zone are open ponds, the liquid level of the second reaction zone has a higher surface elevation than that in the first reaction zone. Thus, the aqueous stream leaving the second reaction zone can flow by gravity into the first reaction zone. In one specific exemplary embodiment, the liquid level of the first reaction zone (e.g., a pond) is between about 10 cm and 500 cm and the liquid level of the second reaction zone (e.g., a pond) is at least 1 cm less than the liquid level in the first reaction zone or more than about 2 cm less than the liquid level in the first reaction zone whereas the liquid level in the second reaction zone is within the range of about 9 cm and 499 cm. In a specific exemplary embodiment, the liquid level of the first reaction zone (e.g., a pond) is between about 10 cm and 400 cm and the liquid level of the second reaction zone (e.g., a pond) is about 15 to 500 cm. In another exemplary embodiments, the surface-to-volume ratio of the second reaction zone is lesser (e.g., smaller or lower) than the surface-to-volume ratio of the first reaction zone. In exemplary embodiments, the surface-to-volume ratio of the first reaction zone ranges between about 0.1 and 200/meters. In exemplary embodiments, when the first reaction zone is an open pond and the second reaction zone is an open pond, the second reaction zone has a greater liquid level than the first reaction zone and/or a lesser surface-to-volume ratio than the first reaction zone.

In exemplary embodiments, the systems or methods described herein are operated under continuous flow conditions optionally through all transport pipes and through the reaction zones and harvester. The systems or methods described herein can also be operated under non-continuous flow conditions e.g., through one or more of the transport pipes.

In other embodiments, the systems described herein are at least partially gravity flow systems, wherein gravity flow of the material is provided from the first reaction zone to the harvesting zone, from the harvesting zone to the second reaction zone, and/or from the second reaction zone to the first reaction zone. In exemplary embodiments, the material from the first reaction zone to the harvesting zone, the material from the harvesting zone to the second reaction zone, and/or the material from the second reaction zone to the first reaction zone is/are transported through the transport lines by gravity flow.

In addition to describing systems for efficient glycerol removal, another aspect of the present disclosure relates to methods of removing glycerol from an aqueous glycerol stream produced from an aquaculture system.

These methods may include the following steps: (i) culturing a first population of microorganisms that produce glycerol in a first reaction zone of the aquaculture system, thereby providing an aqueous stream containing a microbial biomass material that includes glycerol; (ii) inputting the aqueous stream containing the biomass material and glycerol to a harvesting zone; (iii) separating the biomass material in the aqueous stream from the glycerol in the harvesting zone, thereby providing an aqueous glycerol stream; (iv) supplying the aqueous glycerol stream to a second reaction zone containing a second population of microorganisms that metabolize glycerol; and/or (v) removing glycerol from the aqueous glycerol stream by cultivating the second population of microorganisms in the second reaction zone with the aqueous glycerol stream, thereby providing an enriched aqueous stream containing glycerol metabolites.

In addition to describing systems for efficient glycerol removal, another aspect of the present disclosure relates to a method for producing an aqueous glycerol stream having a dissolved glycerol concentration from about 0.1 to about 20000 ppm, the method can include at least one or more of the following steps: culturing a first population of microorganisms that produce glycerol in a first reaction zone, thereby providing an aqueous stream containing a microbial biomass material that includes glycerol; inputting the aqueous stream containing the microbial biomass that includes glycerol to a harvesting zone; and separating the microbial biomass from the glycerol in the harvesting zone.

It has been surprisingly discovered that the methods of the present disclosure and the aquaculture systems including a second reaction zone, containing microorganisms (e.g., heterotrophic and/or mixotrophic microorganisms) that can metabolize glycerol, for example, into easily purified biological products downstream from a harvesting zone can effectively remove glycerol from an aqueous stream without the need of expending the large amounts of energy as seen e.g., with current distillation processes.

It is also surprising that the systems discovered by the inventors of the present disclosure can also be used to establish a continuous method or aquaculture system, thereby saving on resources and reducing environmental waste.

### EXAMPLES

The present disclosure will be described in more detail with reference to the following Examples, which shows exemplary embodiments in accordance with the present disclosure. The present disclosure is not limited to these exemplary embodiments.

### Example 1

Algae cells were separated from a cultivation medium derived from the cultivation of algae. The resulting effluent containing glycerol from which algae cells were separated was put to a shake flask. Optionally the effluent was sterilized in an autoclave before putting it into a sterile shake flask. Inoculum of halotolerant organisms was added to a 250 ml shake flask containing 50 ml effluent and flasks were put to an incubator operated at 28 °C and 200 rpm shaking for 5, 7 and 14 days. Glycerol concentration in the effluent (culture medium) is determined before the incubation and after the incubation.

After the incubation period, the cells are studied under a microscope. In microscopic investigation lipid inclusions can be seen inside the halophilic microorganism cells. The microorganism cells are collected from culture medium in shake flasks by e.g., filtration or centrifugation and the lipid content and composition from the cells is determined. In lipid analysis, triglycerides or polyhydroxyalkonates are detected. The glycerol concentration analysis, performed on the culture medium after cultivation of the halotolerant microorganisms, indicates that the amount of glycerol has decreased during the incubation.

This example indicates that halotolerant microorganisms are able to produce intracellular lipids (e.g., triglycerides or polyhydroxyalkanoates) from the effluent of algae cultivation. This example also indicates that halotolerant microorganisms are able to consume the glycerol in the effluent from algae cultivation. Halotolerant microorganisms are able to utilize glycerol for cell growth and lipid production.

### Example 2: Aquaculture without Polishing Pond

Dunaliella salina algae were grown in an aquaculture system including a one hectare growth pond operating at a salinity of 20 wt % NaCl with a mean hydraulic residence time of seven days. The system was operated in a continuous-flow manner. The liquid level in the growth pond was 40 centimeters. The contents of the pond flowed by gravity over three weirs constructed of high-density polyethylene into three different stress ponds with a total surface area of one hectare (about ⅓ of a hectare for each stress pond). The liquid levels in the stress ponds were maintained at 30 centimeters. The discharge from the stress ponds flowed by gravity over weirs into a sump. A pump was used to transfer the contents of the sump to the harvesting unit as harvester feed. The pump and pipeline were constructed of materials that were not impacted by water that included NaCl. Specifically, a plastic lined canal and high-density polyethylene pipe were used for the transfer. In the harvesting unit, the algal biomass was separated from the growth medium. However, some of the algal biomass and algal components (such as glycerol) were not captured in the harvester and recovered with the algal biomass. Thus, the discharge from the harvesting unit (growth medium recycle) included not only the growth medium but also included algal biomass and algal components that were not captured. When the growth medium was recycled directly to the growth pond (with elevated levels of algal biomass and algal components, e.g., glycerol), the concentration of halotolerant bacteria in the growth pond increased. At night, when the halotolerant bacteria and the algae both respired oxygen, the dissolved oxygen content in the growth pond dropped to zero mg/liter near dawn for multiple days, and the algal culture collapsed.

### Example 3: Aquaculture with Polishing Pond

Dunaliella salina algae were grown in an aquaculture system including one hectare growth pond operating at a salinity of 20 wt % NaCl with a mean hydraulic residence time of seven days, as the system was operated in a continuous-flow manner. The liquid level in the growth pond was 40 centimeters. The contents of the pond flowed by gravity over three weirs constructed of high-density polyethylene into three different stress ponds with a total surface area of one hectare (about ⅓ of a hectare for each stress pond). The liquid levels in the stress ponds were maintained at 30 centimeters. The contents of the stress ponds flowed by gravity over weirs into a sump. A pump was used to transfer the contents of the sump to the harvesting unit as harvester feed. The pump and pipeline were constructed of materials that were not impacted by water that included NaCl. Specifically, a plastic lined canal and high-density polyethylene pipe were used for the transfer. In the harvesting unit, the algal biomass was separated from the growth medium. However, some of the algal biomass and algal components (such as glycerol) were not captured in the harvester and recovered with the algal biomass. Thus, the discharge from the harvesting unit (growth medium recycle) included not only the growth medium but also included algal biomass and algal components that were not captured. The growth medium was recycled through a plastic pipeline into two polishing ponds that were operated in series. These two polishing ponds were each about 0.3 hectares in size and were operated at a liquid level of about 65 and 60 centimeters, respectively. The surface to volume ratio of these ponds were 1.5 to 1.7 per meter. The surface area of the ponds was sufficient to allow sufficient oxygen transport from the atmosphere in order to oxidize the recycled algal biomass and algal components and convert them into CO₂. Contents of the first polishing pond overflowed from a high-density polyethylene weir into the second polishing pond. In turn, the contents of the second polishing pond overflowed from a high-density polyethylene weir into the growth pond. Thus, the two polishing ponds were operated in series. The concentration of algal biomass and algal components was sufficiently low in order to allow good algal growth in the growth pond and in the stress ponds. Thus, the content of the algal biomass and algal components in the growth and stress ponds were sufficiently low so that the dissolved oxygen content in the ponds did not drop to zero mg/liter at night, and the algal culture did not collapse. CO₂ produced by the halotolerant bacteria was consumed by the Dunaliella salina to produce algal biomass that included protein, lipids, and carotenoids.

### Example 4: Aquaculture with Augmented Polishing Pond

Dunaliella salina algae were grown in an aquaculture system including a one hectare growth pond operating at a salinity of 22 wt % NaCl with a mean hydraulic residence time of seven days, as the system was operated in a continuous-flow manner. The liquid level in the growth pond was 40 centimeters. The contents of the pond flowed by gravity over three weirs constructed of high-density polyethylene into three different stress ponds with a total surface area of one hectare (about ⅓ of a hectare for each stress pond). The liquid level in the stress ponds were maintained at 30 centimeters. The contents of the stress ponds flowed by gravity over weirs constructed of high-density polyethylene into a sump. A pump was used to transfer the contents of the sump to the harvesting unit as harvester feed. The pump and pipeline were constructed of materials that were not impacted by water that included NaCl. Specifically, a plastic lined canal and high-density polyethylene pipe were used for the transfer. In the harvesting unit, the algal biomass was separated from the growth medium. However, some of the algal biomass and algal components (such as glycerol) were not captured in the harvester and recovered with the algal biomass. Thus, the discharge from the harvesting unit included not only the growth medium but also included algal biomass and algal components that were not captured. When material was recycled, it was diverted from the harvester discharge through a plastic pipeline into a polishing pond with an aeration unit to accelerate the transport of oxygen into the polishing pond. The polishing pond was about 0.3 hectares in size and was operated at a liquid level of about 65 centimeters. The surface area of the pond, and that generated by the aeration unit, was sufficient to allow efficient oxygen transport from the atmosphere in order to oxidize the recycled algal biomass and algal components, thereby converting them into CO₂. The concentration of algal biomass and algal components was reduced in the polishing pond in order to allow good algal growth in the growth pond and in the stress ponds. Thus, the content of these components in the growth pond and the stress ponds was sufficiently low so that the dissolved oxygen content in the ponds did not drop to zero mg/liter and the algal culture did not collapse. CO₂ produced by the halotolerant bacteria was consumed by the Dunaliella salina to produce algal biomass that included protein, lipids, and carotenoids.

### Example 5: Harvesting with In-Line Polishing via Oxidation

Dunaliella salina algae were grown in an aquaculture system including a one hectare growth pond operating at a salinity of 24 wt % NaCl with a mean hydraulic residence time of ten days, as the system was operated in a continuous-flow manner. The liquid level in the growth pond was 40 centimeters. The contents of the pond flowed by gravity over three weirs constructed of high-density polyethylene into three different stress ponds with a total surface area of one hectare (about ⅓ of a hectare for each stress pond). The liquid levels in the stress ponds were maintained at 30 centimeters. The contents of the stress ponds flowed by gravity over weirs constructed of high-density polyethylene into a sump. A pump was used to transfer the contents of the sump to the harvesting unit. The pump and pipeline were constructed of materials that were not impacted by water that included NaCl. Specifically, a plastic lined canal and high-density polyethylene pipe were used for the transfer. In the harvesting unit, the algal biomass was separated from the growth medium. However, some of the algal biomass and algal components (such as glycerol) were not captured in the harvester and recovered with the algal biomass. Thus, the discharge from the harvesting unit included not only the growth medium but also included algal biomass and algal components that were not captured. The harvester discharge was intimately contacted with ozone as an oxidizing agent in order to oxidize the algal biomass and algal components that were in the stream. After the ozone had contacted the algal biomass to remove more than 90% of the algal biomass and algal components, the stream was discharged into a polishing pond as a backup system to oxidize residual algal biomass and algal components. The polishing pond was about 0.3 hectares in size and was operated at a liquid level of about 65 centimeters. The surface area of the pond and the oxidizer treatment was sufficient to oxidize the recycled algal biomass and algal components, thereby converting them into CO₂. The effluent from the polishing pond was discharged through a high-density polyethylene pipe into the algal growth pond. The concentration of algal biomass and algal components was reduced in the polishing pond in order to allow good algal growth in the growth pond and in the stress ponds. Thus, the content of these components in the growth pond and the stress ponds was sufficiently low so that the dissolved oxygen content in the ponds did not drop to zero and the algal culture did not collapse. CO₂ produced by the halotolerant bacteria was consumed by the Dunaliella salina to produce algal biomass that included protein, lipids, and carotenoids.

### Example 6: Harvesting with In-Line Polishing via UV Radiation

Dunaliella salina algae were grown in an aquaculture system including a one hectare growth pond operating at a salinity of 18 wt % NaCl with a mean hydraulic residence time of six days, as the system was operated in a continuous-flow manner. The liquid level in the growth pond was 40 centimeters. The contents of the pond flowed by gravity over three weirs constructed of high-density polyethylene into three different stress ponds with a total surface area of one hectare (about ⅓ of a hectare for each stress pond). The liquid levels in the stress ponds were maintained at 30 centimeters. The contents of the stress ponds flowed by gravity over weirs constructed of high-density polyethylene into a sump. A pump was used to transfer the contents of the sump to the harvesting unit. The pump and pipeline were constructed of materials that were not impacted by water that included NaCl. Specifically, a plastic lined canal and high-density polyethylene pipe were used for the transfer. In the harvesting unit, the algal biomass was separated from the growth medium. However, some of the algal biomass and algal components (such as glycerol) were not captured in the harvester and recovered with the algal biomass. Thus, the discharge from the harvesting unit included not only the growth medium but also included algal biomass and algal components that were not captured. The harvester discharge was exposed to ultraviolet radiation in order to oxidize the algal biomass and algal components that were in the stream. After irradiating the algal biomass to remove about than 50% of the algal biomass and algal components, the stream was discharged into a polishing pond as a backup system to oxidize residual algal biomass and algal components. The polishing pond was about 0.3 hectares in size and was operated at a liquid level of about 65 centimeters. The surface area of the pond and the oxidizer treatment was sufficient to oxidize the recycled algal biomass and algal components, thereby converting them into CO₂. The effluent from the polishing pond was discharged through a high-density polyethylene pipe into the algal growth pond. The concentration of algal biomass and algal components was reduced in the polishing pond in order to allow good algal growth in the growth pond and in the stress ponds. Thus, the content of these components in the growth pond and the stress ponds was sufficiently low so that the dissolved oxygen content in the ponds did not drop to zero and the algal culture did not collapse. CO₂ produced by the halotolerant bacteria was consumed by the Dunaliella salina to produce algal biomass that included protein, lipids, and carotenoids.

It will be appreciated by those skilled in the art that the present disclosure can be embodied in other specific forms without departing from the spirit or essential characteristics thereof. The presently disclosed embodiments are therefore considered in all respects to be illustrative and not restricted. The scope of the disclosure is indicated by the appended claims rather than the foregoing description and all changes that come within the meaning and range and equivalence thereof are intended to be embraced therein.

1. A method for removing glycerol from an aquaculture system, the method comprising:
culturing a first population of microorganisms that produce glycerol in a first reaction zone, thereby providing an aqueous stream containing a microbial biomass material that includes glycerol;
inputting the aqueous stream containing the microbial biomass material that includes glycerol to a harvesting zone;
separating the microbial biomass from the glycerol in the harvesting zone, thereby providing an aqueous glycerol stream;
supplying the aqueous glycerol stream to a second reaction zone containing a second population of microorganisms that metabolize glycerol; and
removing glycerol from the aqueous glycerol stream by cultivating the second population of microorganisms in the second reaction zone with the aqueous glycerol stream, thereby providing an enriched aqueous stream containing glycerol metabolites;
wherein a ratio of eukaryotic algae cells/ml to prokaryote cells/ml is higher in the first population of microorganisms than in the second population of microorganisms.

2. The method of claim 1, wherein the method comprises recovering the glycerol metabolites from the enriched aqueous stream, thereby providing a depleted aqueous stream.

3. The method of claim 1, wherein the first population of microorganisms contains at least one or more eukaryotic alga, prokaryote, and/or a combination thereof.

4. The method of claim 1, wherein the second population of microorganisms contains at least one or more eukaryotic alga, prokaryote, and/or a combination thereof.

5. The method of claim 1, wherein a ratio of eukaryotic algae cells/ml in the first population of microorganisms to eukaryotic algae cells/ml in the second population of microorganisms is more than 1, and/or a ratio of prokaryote cells/ml in the first population of microorganisms to prokaryote cells/ml in the second population of microorganisms is less than 1.

6. The method of claim 1, wherein the aquaculture system is a continuous flow reactor system.

7. The method of claim 1, wherein the method comprises injecting an effluent stream into the first reaction zone from the second reaction zone, thereby providing a continuous glycerol removal process.

8. The method of claim 1, wherein at least part of the enriched aqueous stream is supplied to the first reaction zone.

9. The method of claim 1, wherein the glycerol metabolites are selected from a group consisting of water, carbon dioxide, biomass, proteins, lipids, carotenoids, polymers, alcohols, acids, diols and any combinations thereof.

10. The method of claim 1, wherein the aqueous stream containing the microbial biomass and glycerol comprises a salinity from about 5 wt % to about 27 wt %.

11. The method of any claim 1, wherein the first reaction zone is at least one or more of an open pond, a bioreactor, and/or a combination thereof.

12. The method of claim 1, wherein the second reaction zone is at least one or more of an open pond, a bioreactor, and/or combinations thereof.

13. The method of claim 1, wherein when the first reaction zone is an open pond and the second reaction zone is an open pond, the second reaction zone possesses a greater liquid level than the first reaction zone and/or a lesser surface-to-volume ratio than the first reaction zone.

14. The method of claim 1, wherein the culturing comprises enhancing growth conditions in the first reaction zone and/or enhancing the cultivating in the second reaction zone.

15. The method of claim 14, wherein the enhancing growth conditions in the first reaction zone comprises increasing/decreasing light availability of the first reaction zone, lowering a concentration of dissolved glycerol in the first reaction zone and/or decreasing turbidity of the first reaction zone.

16. The method of claim 1, wherein separating the microbial biomass from the glycerol in the harvesting zone comprises rupturing the microbial biomass to release the glycerol.

17. The method of claim 1, wherein the harvesting zone includes an adsorptive bubble separation unit.

18. The method of claim 1, wherein the harvesting zone comprises at least one or more of a centrifugation unit, a flocculation unit, a sedimentation unit, a filtration unit, and/or any combination thereof.

19. The method of claim 1, wherein the aqueous glycerol stream possesses a dissolved glycerol concentration from 0.1 to 20000 ppm.

20. The method of claim 1, wherein the aqueous glycerol stream possesses a salinity from about 5 wt % to about 27 wt %.

21. A composition produced according to the methods of claim 1, the composition comprising glycerol metabolites recovered from at least the enriched aqueous stream and/or the aqueous glycerol stream, wherein the composition possesses the glycerol metabolites in a concentration that ranges from about 1 wt % to about 90 wt % of a total weight of the composition.

22. An enriched aqueous stream containing glycerol metabolites obtained by the method according to claim 1, wherein the enriched aqueous stream possesses a dissolved glycerol concentration that ranges from about 1% to about 90% of the aqueous glycerol stream's dissolved glycerol concentration.

23. The enriched aqueous stream containing glycerol metabolites of claim 22, comprising a salinity between about 5 wt % and saturation.

24. The enriched aqueous stream containing glycerol metabolites of claim 22, comprising a ratio of eukaryotic algae cells to prokaryote cells less than 1.

25. The enriched aqueous stream containing glycerol metabolites of claim 22, comprising any one or more of the following:
a total organic carbon amount ranging from 10 to 400 mg/L;
a chemical oxygen demand amount ranging from 30 to 900 mg/L;
a phosphorus amount ranging from 0.3 to 300 mg/L; or
a nitrogen amount ranging from 0.5 to 400 mg/L.

26. An aquaculture system, wherein glycerol is removed from the system, the system comprising:
a first reaction zone including a first population of microorganisms that produce glycerol;
a harvesting zone in communication with the first reaction zone, the harvesting zone including a harvester; and
a second reaction zone in communication with the harvesting zone, the second reaction zone including a second population of microorganisms that metabolize glycerol;
wherein a ratio of eukaryotic algae cells/ml to prokaryote cells/ml is higher in the first population of microorganisms than in the second population of microorganisms.

27. The system of claim 26, wherein the second reaction zone is in communication with the first reaction zone, thereby providing a continuous flow reactor system.

28. The system of claim 26, wherein the communication between the harvesting zone and first reaction zone is provided by at least one or more of a pipe, a tube and/or a canal.

29. The system of claim 28, wherein the pipe or tube is formed from at least one or more of plastic, concrete, clay and/or any material resistant to salt corrosion.

30. The system of claim 28, wherein the canal possesses a cover.

31. The system of claim 26, wherein the communication between the harvesting zone and second reaction zone is provided by at least one or more of a pipe, a tube and/or a canal.

32. The system of claim 26, wherein the system is at least partially a gravity flow system, wherein gravity flow is provided from the first reaction zone to the harvesting zone, from the harvesting zone to the second reaction zone, and/or from the second reaction zone to the first reaction zone.

33. The system of claim 26, wherein the system comprises at least one or more of the following:
a first recovery line in communication with the harvesting zone, the first recovery line being configured to recover microbial biomass material from the harvesting zone; and/or
a second recovery line in communication with the second reaction zone, the second recovery line being configured to recover glycerol degradation products or metabolites from the second reaction zone.

34. The system of claim 26, wherein the first reaction zone is at least one or more of an open pond, a bioreactor, and/or a combination thereof.

35. The system of claim 26, wherein the second reaction zone is a biological polishing system, the polishing system being at least one or more of an open pond, a bioreactor, and/or a combination thereof.

36. The system of claim 35, wherein when the first reaction zone is an open pond and the second reaction zone is an open pond, the second reaction zone has a greater liquid level than the first reaction zone and/or a lesser surface-to-volume ratio than the first reaction zone.

37. The system of claim 26, wherein:
the first population of microorganisms contains at least one or more of eukaryotic alga, prokaryote, and/or combinations thereof;
the second population of microorganisms contains at least one or more of eukaryotic alga, prokaryote and/or combinations thereof;
the second population of microorganisms contains more prokaryote cells/ml than the first population of microorganisms; and/or
the first population of microorganisms contains more eukaryotic algae cells/ml than the second population of microorganisms.

38. The system of claim 26, wherein a ratio of eukaryotic algae cells/ml in the first population of microorganisms to eukaryotic algae cells/ml in the second population of microorganisms is more than 1; and/or a ratio of the prokaryote cells/ml in the first population of microorganisms to the prokaryotes in the second population of microorganisms is less than 1:1.

39. The system of claim 26, wherein the system further comprises
an aquaculture pretreatment unit configured for conditioning an aqueous medium with at least algal nutrients to obtain the aquaculture medium, wherein the aquaculture pretreatment unit is arranged to feed the aquaculture medium to the one or more first reaction zones optionally by gravity feed; and/or
one or more ponds for crystallizing salt, optionally in communication with the second reaction zone.

40. Use of the system of claim 26 for removing glycerol from an aqueous glycerol stream.

41. The method of claim 1, wherein the aqueous glycerol stream is contacted with an oxidizing agent and/or exposed to ultraviolet radiation.
